(19) United States
(12) Patent Application Publication
(10) Pub. No.: US 2023/0226308 A1
(43) Pub. Date: Jul. 20, 2023

(54) Personal Thermal Stability Control

(71) Applicant: Amira Health Inc., San Francisco, CA (US)

(72) Inventors: Claire Morgan Traweek, Cambridge, MA (US); Daniel Tortora Wiest, Torrance, CA (US); Emilio Sison, Cambridge, MA (US); Felipe Radovitzky, Cambridge, MA (US); Loewen Katherine Cavill, Milford, OH (US)

(21) Appl. No.: 18/007,117

(22) PCT Filed: Jul. 30, 2021
(86) PCT No.: PCT/US2021/043948

§ 371 Date: Jan. 27, 2023

Related U.S. Application Data
(60) Provisional application No. 63/058,625, filed on Jul. 30, 2020.
(60) Provisional application No. 63/140,826, filed on Jan. 23, 2021.

(51) Int. Cl. A61M 21/00

(52) U.S. Cl. CPC A61M 21/00; A61M 2021/0066; A61M 2205/3606; A61M 2205/13; A61M 2205/3303; A61M 2230/65; A61M 2230/005; A61M 2205/42; A61M 2205/3569; A61M 2210/1003; A61M 2205/362; A61M 2205/366

(57) ABSTRACT

Personal thermal stability control herein provides for personalized temperature regulation dependent upon biometric sensor feedback, sleep stage, and so on, particularly for sleeping users, predicting and preemptively responding to fluctuations indicative of thermal stability, resulting from such things as transitions between sleep stages, hot flashes, night sweats, and general thermal instability. Specifically, in one embodiment, a system herein may comprise: an on-demand cooling system; one or more biometric sensors configured to monitor one or more corresponding indicators of thermal stability of a user; and a controller configured to: a) receive the one or more corresponding indicators of thermal stability of the user; b) predict an onset of a thermal instability of the user based on the one or more corresponding indicators of thermal stability of the user; and c) activate the on-demand cooling system to counteract the predicted onset of a thermal instability of the user.

### RELATED APPLICATIONS

The present application is a U.S. national entry of International App. No. PCT/US2021/043948 filed Jul. 30, 2021, entitled “PERSONAL THERMAL STABILITY CONTROL”, which claims the benefit and priority of U.S. Provisional App. No. 63/058,625 filed on Jul. 30, 2020, and U.S. Provisional App. No. 63/140,826 filed Jan. 23, 2021, both entitled “NOVEL SLEEP TEMPERATURE OPTIMIZATION SYSTEM ACTIVELY CONTROLLED BY WEARABLE SENSOR SUITE”, all of which are hereby incorporated by reference in their entirety.

### TECHNICAL FIELD

The present disclosure relates generally to thermal control technology, and, more particularly, to personal thermal stability control.

### BACKGROUND

Personal thermal stability is generally based on a number of factors, such as ambient temperature, insulation from ambient temperature (e.g., clothing, blankets, etc.), and body temperature control (e.g., sweating, blood flow, etc.). For instance, a healthy body typically functions best at an internal temperature of about 98.6° F. (37° C.), and humans constantly adapt their temperature to environmental conditions, different physical activities, and at various times throughout the day. Internal body temperature is regulated by the hypothalamus in the brain, which compares the body's current temperature to the desired temperature (e.g., 98.6° F.), such that if the temperature is too low, the hypothalamus instructs the body to generate and maintain heat. If, on the other hand, the current body temperature is too high, heat is given off and/or sweat is produced to cool the skin.

Sleep temperature, in particular, plays a critical role in increasing the quality and efficiency of rest which can have large effects on the health of an individual. Unfortunately, however, many individuals suffer from poor personal thermal stability control during sleep. For instance, one of the most common effects of menopause is disrupted sleep due to hormonal transitions manifested as hot flashes and night sweats. Additionally, many athletes, students and other brain workers, sufferers of fevers and other diseases, and so on, may also benefit from increasing the advantages associated with personal thermal stability control during sleep.

### SUMMARY

According to one or more embodiments of the disclosure, devices, systems, and techniques introduced herein relate to personal thermal stability control. In particular, the techniques herein provide for personalized temperature regulation dependent upon biometric sensor feedback, sleep stage, and so on, particularly for sleeping users. For example, an illustrative embodiment of the present invention may comprise a temperature stabilization system utilizing a wearable sensor, an on-demand cooling system (e.g., a hydronically cooled mattress pad), and a predictive controller. The techniques herein may track biometric indicators in real time to predict and preemptively respond to fluctuations indicative of thermal stability, resulting from such things as transitions between sleep stages, hot flashes, night sweats, and general thermal instability.

Specifically, in one embodiment, a system herein may comprise: an on-demand cooling system; one or more biometric sensors configured to monitor one or more corresponding indicators of thermal stability of a user; and a controller configured to: a) receive the one or more corresponding indicators of thermal stability of the user; b) predict an onset of a thermal instability of the user based on the one or more corresponding indicators of thermal stability of the user; and c) activate the on-demand cooling system to counteract the predicted onset of a thermal instability of the user.

Other embodiments of the present disclosure may be discussed in the detailed description below, and the summary above is not meant to be limiting to the scope of the invention herein.

### BRIEF DESCRIPTION OF THE DRAWINGS

The embodiments herein may be better understood by referring to the following description in conjunction with the accompanying drawings in which like reference numerals indicate identically or functionally similar elements, of which:

FIG. 1 illustrates an example simplified system for providing personal thermal stability control in accordance with one or more embodiments described herein;

FIG. 2 illustrates a specific example simplified system for providing personal thermal stability control in accordance with one or more embodiments described herein;

FIGS. 3A-3B illustrate example simplified wearable sensor devices for use with providing personal thermal stability control in accordance with one or more embodiments described herein;

FIGS. 4A-4D further illustrate example simplified wearable sensor devices for use with providing personal thermal stability control in accordance with one or more embodiments described herein;

FIG. 5 illustrates an example simplified sensor filter circuit for use with providing personal thermal stability control in accordance with one or more embodiments described herein;

FIGS. 6A-6B illustrate example alternative simplified wearable sensor devices for use with providing personal thermal stability control in accordance with one or more embodiments described herein;

FIG. 7 illustrates an example simplified cooling system for use with providing personal thermal stability control in accordance with one or more embodiments described herein;

FIGS. 8A-8B illustrate an example simplified control hub for use with providing personal thermal stability control in accordance with one or more embodiments described herein;

FIGS. 9A-9C illustrate examples of simplified cooling pads for use with providing personal thermal stability control in accordance with one or more embodiments described herein;

FIG. 10 illustrates an example simplified user device and associated graphical user interface (GUI) for use with providing personal thermal stability control in accordance with one or more embodiments described herein;

FIGS. 11A-11D illustrate examples of sensor data and computation associated with providing personal thermal stability control in accordance with one or more embodiments described herein;

FIG. 12 illustrates an example simplified procedure for providing personal thermal stability control in accordance with one or more embodiments described herein;

FIG. 13 illustrates an example simplified device for providing personal thermal stability control in accordance with one or more embodiments described herein; and

FIG. 14 illustrates an example of a benefit of providing personal thermal stability control in accordance with one or more embodiments described herein.

### DETAILED DESCRIPTION

As noted above, personal thermal stability is generally based on a number of factors, where humans constantly regulate their internal body temperature. Further, sleep temperature, in particular, plays a critical role in increasing the quality and efficiency of rest which can have large effects on the health of an individual. Thermoregulation during sleep is critical to minimizing disruptions and maximizing its associated benefits. Low sleep quality and disruptions can have serious negative effects on both an individual's productivity and health, with studies showing impaired immunity and memory, and increased risk of weight gain and heart disease accompanying the lack of sleep.

As also noted above, however, many individuals suffer from poor personal thermal stability control during sleep. For instance, although the symptoms and experiences associated with menopause vary drastically from woman to woman, one of the most common effects of menopause is disrupted sleep (e.g., 81% of women), where much of this disruption is the result of temperature instability arising due to hormonal transitions manifested as hot flashes and night sweats. A hot flash is the sudden feeling of warmth in the upper body, which is usually most intense over the face, neck, and chest, often causing sweating and reddening of the skin. Hot flashes are most commonly caused by changing hormone levels before, during, and after menopause, with most research suggesting that hot flashes occur when decreased estrogen levels cause a woman's hypothalamus to become more sensitive to slight changes in body temperature. That is, when the hypothalamus thinks the body is too warm, it starts a chain of events—a hot flash—to cool the body down.

Therefore, to counter the prevalence of temperature instability, whether menopause-related or otherwise, the techniques herein provide for personalized temperature regulation dependent upon biometric sensor feedback, sleep stage, and so on, particularly for sleeping users. For example, as described in greater detail below, an illustrative embodiment of the present invention may comprise a temperature stabilization system utilizing a wearable sensor, an on-demand cooling system (e.g., a hydronically cooled mattress pad), and a predictive controller. The techniques herein may track biometric indicators in real time to predict and preemptively respond to fluctuations indicative of thermal stability, resulting from such things as transitions between sleep stages, hot flashes, night sweats, and general thermal instability. Notably, the techniques herein are applicable to more than just menopausal women, also providing optimal recovery for athletes, greater mental clarity for students and other brain workers, a more restful sleep for sufferers of fevers and other diseases, and so on, thus generally increasing the benefits associated with sleep for anyone.

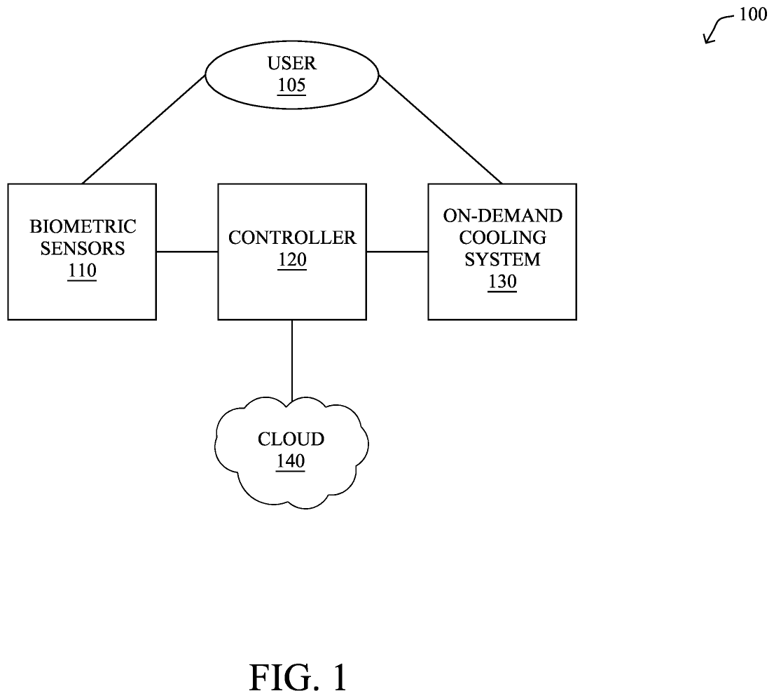

Operationally, and with reference to FIG. 1, an illustrative system 100 herein for a user 105 comprises, at a high level, one or more biometric sensors 110 configured to monitor/collect one or more corresponding indicators of thermal stability of the user, a controller 120 configured to receive the data from the sensors, predict the onset of thermal instabilities (and optionally manual user selections), and to activate an on-demand cooling system 130 to counteract the predicted onset of thermal instability. Optionally, in one embodiment, the system 100 may also comprise a connection to the Cloud 140, such as for computation, data storage, training, and so on, as described further below.

For instance, in one illustrative embodiment, the present invention may comprise an in-bed liquid cooling system (130) that can be controlled both manually or by an automatic sensor system (a combination of sensors 110 and controller 120) to maintain optimal temperature stability during sleep of the user 105.

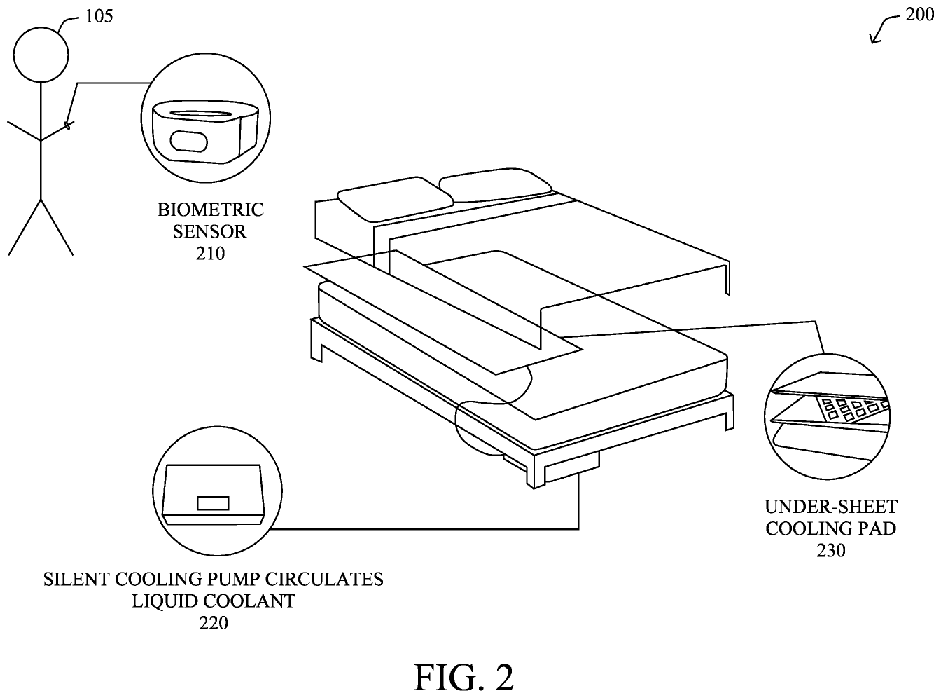

As a specific illustration of this, FIG. 2 shows an example bed-based system 200 herein, where a user 105 can wear a biometric sensor 210 (e.g., a watch, bracelet, etc.) to take readings, which may be relayed (e.g., via WiFi or Bluetooth) to a controller, such as a coolant hub 220 (e.g., a silent cooling pump that circulates liquid coolant) as shown, or else to a smartphone or other device which can relay the data (or the control). The controller (e.g., hub 220 or a phone app, not shown) either predicts hot flashes locally, and/or relays data to the cloud for further analysis or for offloaded prediction (or portions of the prediction) by the cloud. When a hot flash is predicted, or on a manual signal from the wearable is received, the cooling system delivers a cold rush of water to the mattress pad 230 to quickly counteract the effects of the hot flash, accordingly.

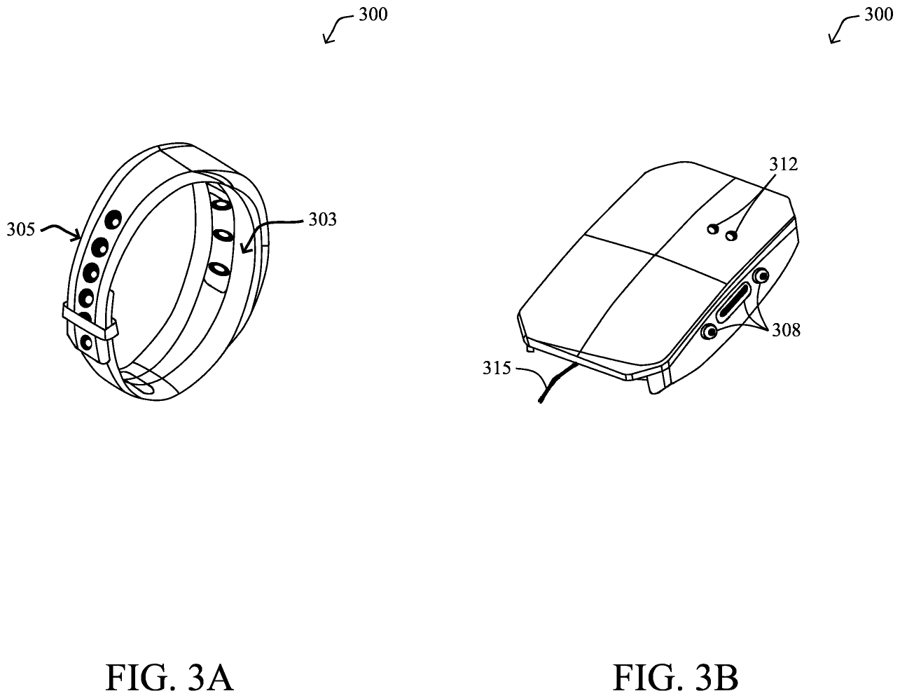

Notably, the wearable sensor generally comprises a wireless device that contains communications hardware and sensors (e.g., electrodes) which monitor the user. As shown in FIG. 3A, an example wearable device 300 (e.g., wristband, watch, anklet, etc.) utilizes one or more biometric sensors 303 against the user's wrist to track both sleep stages and a number of biometric indicators of temperature instability, which may be regularly reported to the controller (e.g., cooling hub). The wearable device/sensor may generally comprise a galvanic skin resistance (GSR) sensor (detecting imperceptible changes in skin moisture—before and during a hot flash, a spike can be seen from this reading, as detailed below), such as flat silver-chloride plated dry electrocardiogram (ECG) electrodes. It is believed that using dry/non-gel electrodes is more comfortable for the user, but alternative embodiments may include metal snap electrodes, or other types of electrodes as will be understood by those skilled in the art.

Additional and/or alternative biometric sensors may also be used with the wearable device, such as photoplethysmography (PPG) sensors (as described below), other sensors that monitor blood flow, vasodilation, skin conductance, sweatiness, etc., as well as other general sensors, such as an accelerometer, gyroscope, and a temperature sensor (user and/or ambient), among others. Accelerometers, for instance, can detect when the user rolls over, etc., and also gives information on the sleep stage of the user (e.g., REM, deep sleep, waking, etc.), which is useful for predicting sleep cycles, for example.

Illustratively, in FIG. 3A, the sensors 303 are located on the inside of the wrist band with contact sensor surfaces planar to the rear surface of the wearable in order to ensure consistent contact by being secured to the user. While the sensor surface 303 is curved to match a general user's body profile, an adjustable band sizing mechanism 305 allows for customizable and secure fit for all wrist sizes.

In FIG. 3B, the illustrative wearable device 300 may also have one or more buttons 308 for manual control and/or feedback (e.g., temperature up/down, select, toggle, on/off, etc.), as well as one or more output indicators 312, such as lights, LEDs, a display, an LCD display, a GUI, and so on. Optionally, in certain embodiments, additional sensors (e.g., on the wristband, in a glove, etc.) may utilize an ultra flexible wire 315 (e.g., silicone) or flexible circuit board layer to pass signals from the band to a printed circuit board (PCB) in the casing of the device 300 (not specifically shown).

It is important to note that the techniques herein specifically address noise minimization in sensor data. Noise can come from changes in pressure, from movement leading to contact surface area changes, from environmental resistance contamination and non-hot-flash sources of conductance changes, such as emotional sweating, and so on. That is, certain aspects of the embodiments herein are specifically designed to counteract these sources of noise, accordingly.

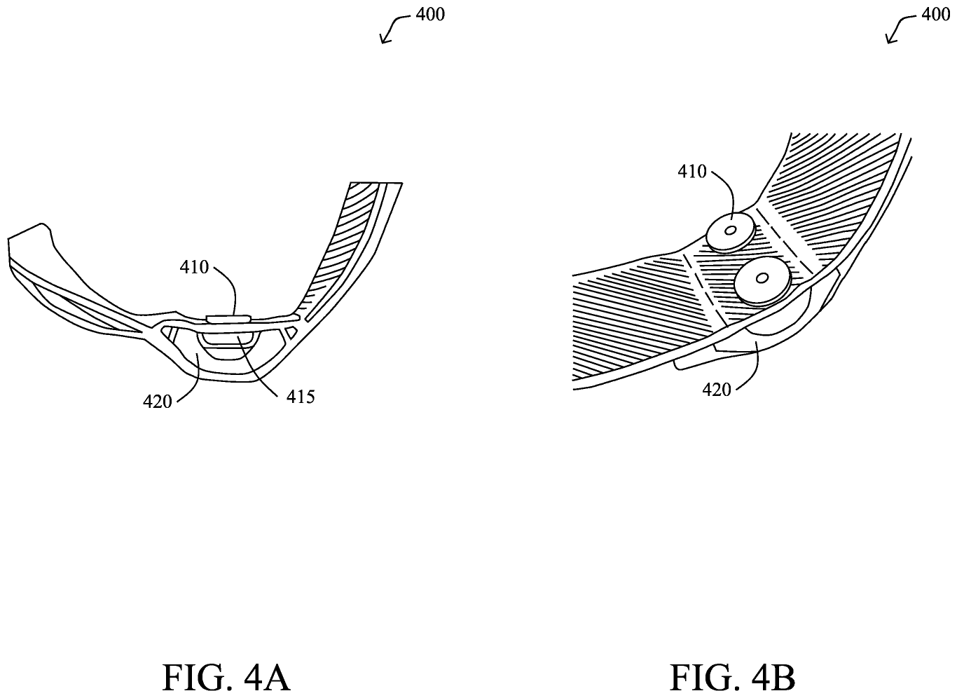

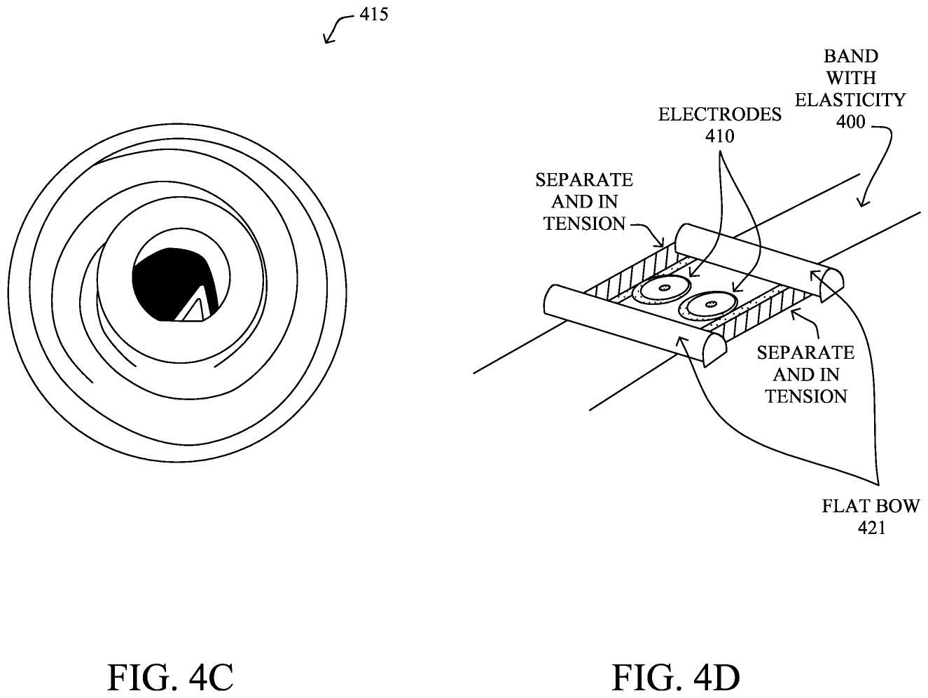

For instance, as an alternative embodiment, FIGS. 4A-4D show a wearable device 400 where the sensors 410 (e.g., electrodes) are mounted in the band with a bracket 420 which holds the electrodes tight against the user's skin. In particular, this arrangement places the sensors on the palm side of the wrist, which often produces better measurements. The sensors may communicate with the PCB of the device 400 through wires (similar to wires 315 above) (e.g., which may be attached with wire glue). The wires may be woven weaved into the band or stitched to the band, generally in a manner such that so they do not don't get caught on objects, particularly at night. The band is generally elastic, creating a springiness that is essential to maintaining skin contact with the sensors (electrodes) 410, and, as shown in FIGS. 4A and 4C, the sensors may be held in place on the band with 3D printed snaps 415.

One issue that many skin conductivity sensors experience is that pressure has a substantial effect on measurements, because the area of skin coming into contact with an electrode directly affects the reading. That is, if pressure is applied between the skin and the sensors, the skin's normally textured” surface presses flat against the electrode, resulting in a greater area of contact, and thus greater conductivity readings. As such, it is important to maintain an even level of pressure, otherwise the variance in readings may adversely affect the algorithms herein. According to the techniques herein, shown in FIGS. 4A and 4B, specifically, by having a rigid “bow shaped” bracket 420, the consistent “springiness” pressure of the band at the location of the sensors 410 reduces noise of the readings, such as by shielding the electrodes from external pressure (for instance, someone rolling over). Alternatively, a “flat bow” 421 may be used to provide a bracket that holds the two opposing sides of the bow separate and in tension for the mounted electrodes/sensors 410, accordingly, with a flatter/slimmer profile than a bow bracket's rigid “U” shape. In either embodiment, the electrodes, together with the elasticity of the band, apply a more consistent force to the skin.

While the bow design above helps, there are still instances where noise is produced at the sensors (e.g., moving/flexing the wrist changes the profile seen on sensors). While software can remove some noise, more or less separating these effects as they are seen (e.g., correlated with motion detection or otherwise), a capacitance-based circuit may be used to better account for surface area changes. That is, specific embodiments the techniques herein may use capacitance across the electrodes as a way to account for surface area changes.

Said differently, since measuring resistance across the electrodes (as currently measured with GSR sensors) is directly proportional to the skin-contact area with the electrodes, any change in this measurement could equally come from changes in contact area just as much as the amount of sweat. On the other hand, by using capacitance, which is almost completely governed by surface area and not sweat, the techniques herein can cancel out the surface-area effect. This would allow the system of the present disclosure to better estimate the skin's specific conductance regardless of shifts in the amount of skin touching the electrodes. Preliminary tests have shown that capacitance does change a significant and repeatable amount in response to the changes in surface area that would be normal when the bracelet is being used.

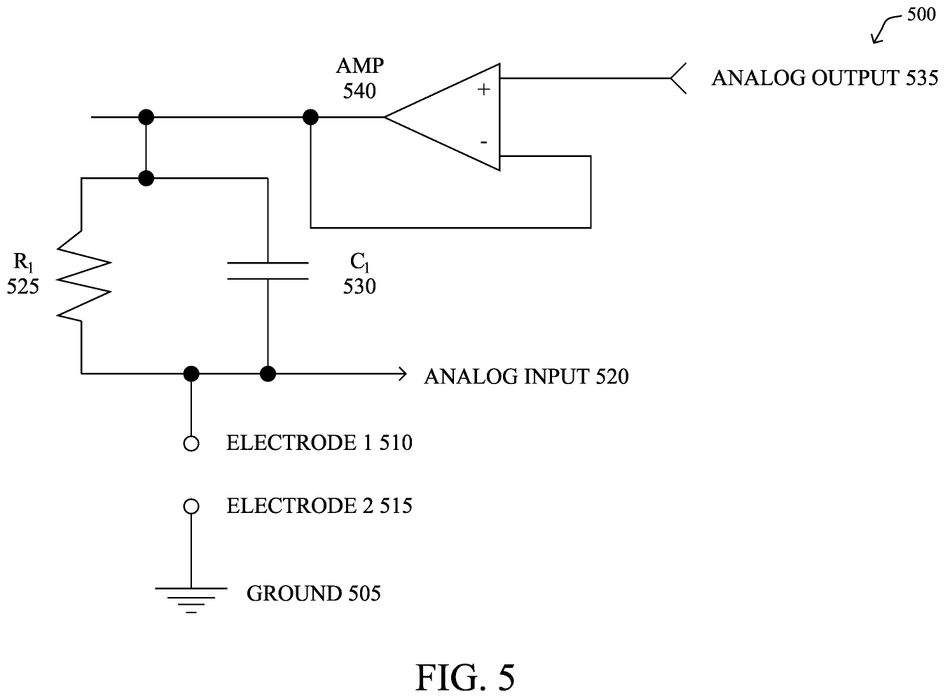

FIG. 5 is an example circuit 500 that could be used to measure both resistance and conductance at the electrodes 1 (510) and 2 (515). By varying the microprocessor's named analog output 535 (with associated amp 540), and measuring the time-dependent response of the named analog input 520, both conductance and resistance can be found, as may be appreciated by those skilled in the art. Example set values that may be used would be 1 megaohm for resister R1 (525), and 1 picofarad for capacitor C1 (530). Alternatively, other impedance-measurement integrated circuits may be used to obtain these values. Using the touch/capacitive sensing in this manner, i.e., the level of contact of the capacitive circuit being completed, the resistance and conductance values may be used to correspondingly cancel out changes in skin contact/pressure, accordingly.

Moreover, studies have shown that vasodilation (blood vessel dilation) is a strong really good indicator of coming hot flashes. In some cases, vasodilation indicators for hot flashes can even precede conductance indicators. However, the standard and most reliable method to measure vasodilation in a clinical setting is with a large laser Doppler flowmeter machine, which at current scale cannot be placed in a bracelet. Though use of a miniaturized laser Doppler flowmeter is conceived herein, one alternative sensor to measure vasodilation is a photoplethysmography (PPG) sensors. PPG sensors, for example with red, green, and infrared light, can be mapped to vasodilation/laser Doppler flowmeter readings and thus vasodilation through filters and machine learning. That is, by monitoring PPG readings over time and correlating such PPG readings with vasodilation/laser Doppler flowmeter readings, the PPG sensors may then be used to map its readings to the more powerful vasodilation/laser Doppler flowmeter readings, accordingly. (Note that PPG sensors may be in place of GSR sensors, or in addition to the GSR readings for greater accuracy and predictions further in advance.)

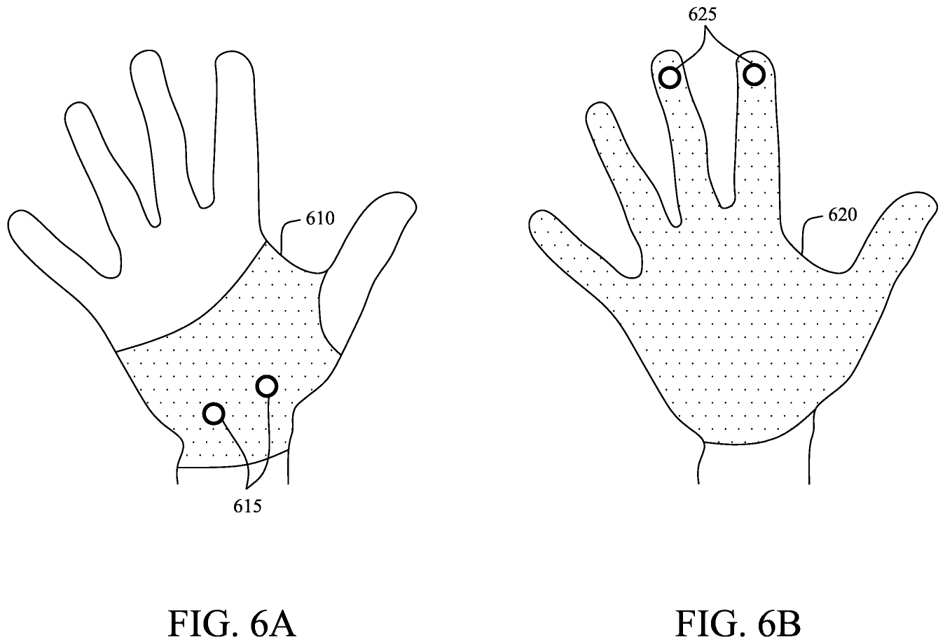

FIGS. 6A-6B show alternative designs of sensor placement, such as a glove (e.g., partial glove) 610 in FIG. 6A with palm electrodes 615, glove 620 in FIG. 6B with finger electrodes 625, or still others, such as a sternal sensor hub, or an eye mask, etc. Also, as alternatives to gloves as shown, other configurations, such as straps, fewer fingers, and so on, may also be used herein, and those shown herein are merely examples.

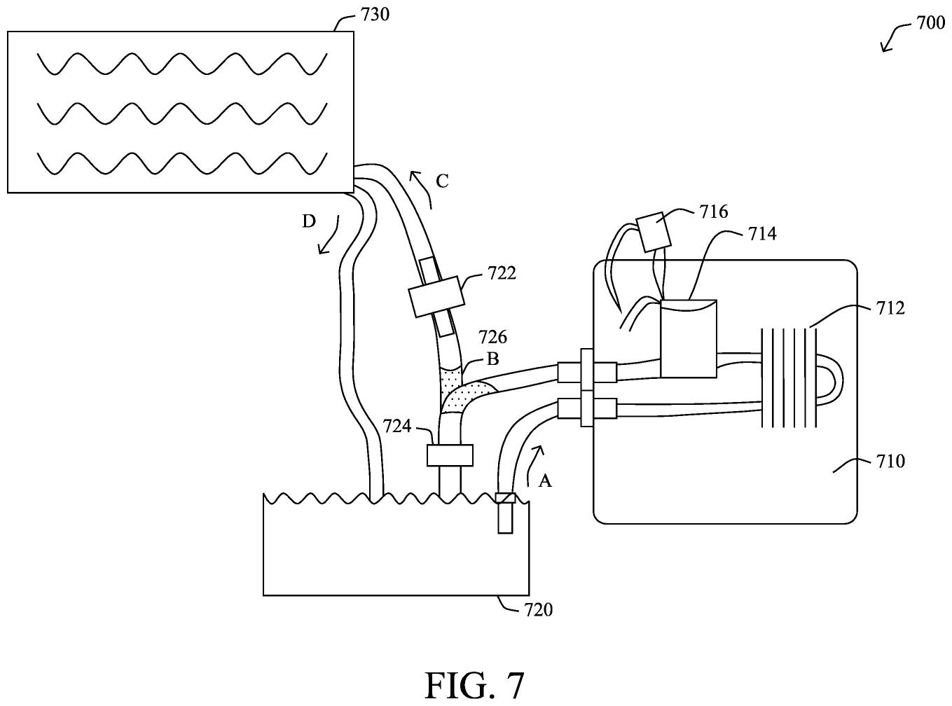

Turning now to the cooling system, FIG. 7 illustrates an example configuration of a system 700 that may be used in accordance with one or more embodiments herein. Specifically, in the example system, though other techniques may be used herein, a flow control hub/chiller 710 may use one or more Peltier coolers 712 (e.g., coupled with a liquid heat exchanger), where one or more pumps 714 (e.g., controlled via relay 716) and valves 722 and 724 route the liquid through either the reservoir 720 only, or the mattress pad 730 (e.g., and then the reservoir). Valves 722 and 724 (e.g., solenoids) control flow of the liquid through a splitter 726, to either travel in flow A-B (e.g., 722 is closed, 724 is open), or in flow A-B-C-D (724 is closed, 722 is open). Other flow control configurations and directions may be made and used herein, and the one shown in FIG. 7 is one non-limiting example. Also note that the flow need not be binary “on or off”, but may also have various levels of speed, mixtures (e.g., some flow to pad, some flow to reservoir), and so on, based on desired cooling output from the system, accordingly.

Note that the cooling pad 730 in this configuration is not constantly in circulation or being cooled. Instead, contrary to conventional cooling mattress pads that only continuously circulate water, the techniques herein may actuate the pumps and valves on-demand to direct flow when needed (e.g., quickly when a hot flash is coming) from a reservoir 720 of pre-cooled water. That is, by keeping a reservoir of cool water as a part of the circulation, a significant amount of cold water may be quickly pumped through the mattress pad, with the cool temperature being adequately maintained by the cooling system for the duration of the need (e.g., duration of the hot flash). This is particularly important for the generally small form factor of the cooling system herein.

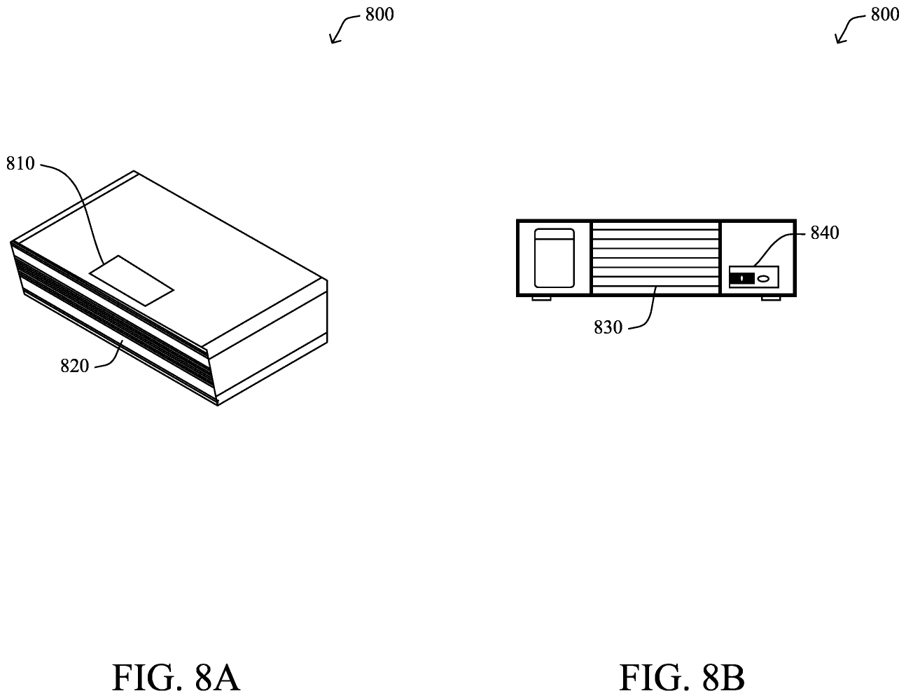

For instance, with reference to FIGS. 8A-8B, an example under-bed cooling hub 800 may comprise hardware for both cooling and pumping, as shown above in FIG. 7. The cooling hub unit may contain an interface 810 for user interaction and adjustment (e.g., a display, controls, GUI, etc.), or else circuity to communicate (e.g., wirelessly) with a user-facing interface (e.g., a phone, watch, remote, etc.). The constrained height of the hub allows it to fit underneath standard bed frames, where a front intake grille 820 in FIG. 8A allows airflow passage from the front (and outward) face of the hub through the heat exchange system within the hub, venting to an aft air exhaust grille 830 on the rear of the hub in FIG. 8B (and thus to the underside of the bed). The cooling unit 800 also has ports 840 to connect to wall power and to connect hydronic cooling tubing to the mattress pad, accordingly (interface not specifically shown).

The illustrative hydronically cooled mattress pad, cover, or layer, etc. referenced above facilitates heat exchange between the user and a circulating fluid. A phase change cooling system, such as those shown above, may interface directly with the circulating fluid loop to pass chilled water through corresponding pathways of the mattress pad in order to provide cooling to the user and thus extract heat from the user when necessary (e.g., during a hot flash). An example mattress pad is a multi-layer topper designed to go on top of the mattress or on top of the mattress cover, and below the sheets, though in alternative embodiments may be a part of the bed itself, part of a blanket or comforter, and so on. In general, the pad lays under one person (e.g., half a queen or king bed). (Note that while air cooling is an option herein, the directed pad design herein provides a significant advantage by reducing the impact of cooling on any partners, and providing faster, more direct cooling to where it is needed most.)

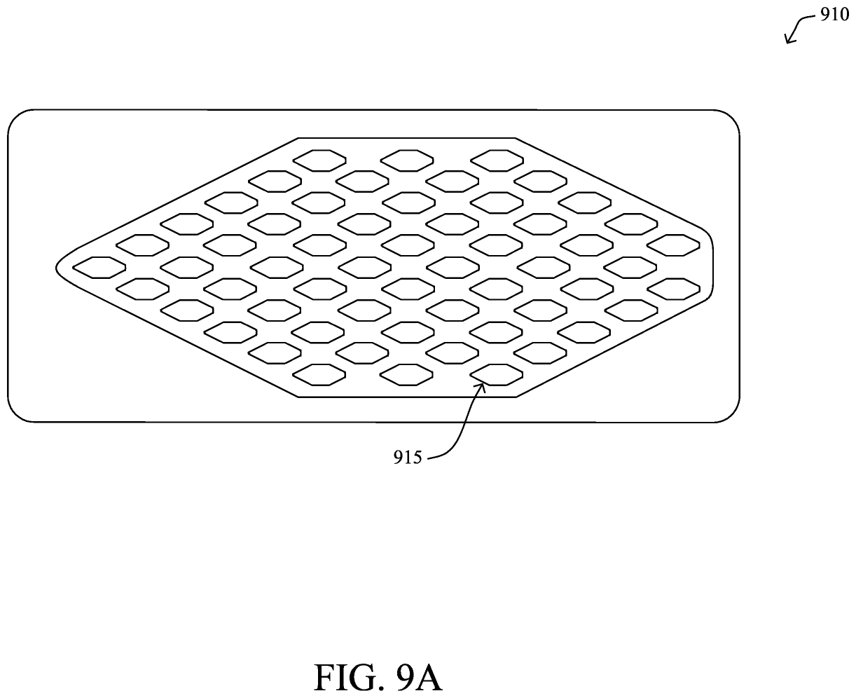

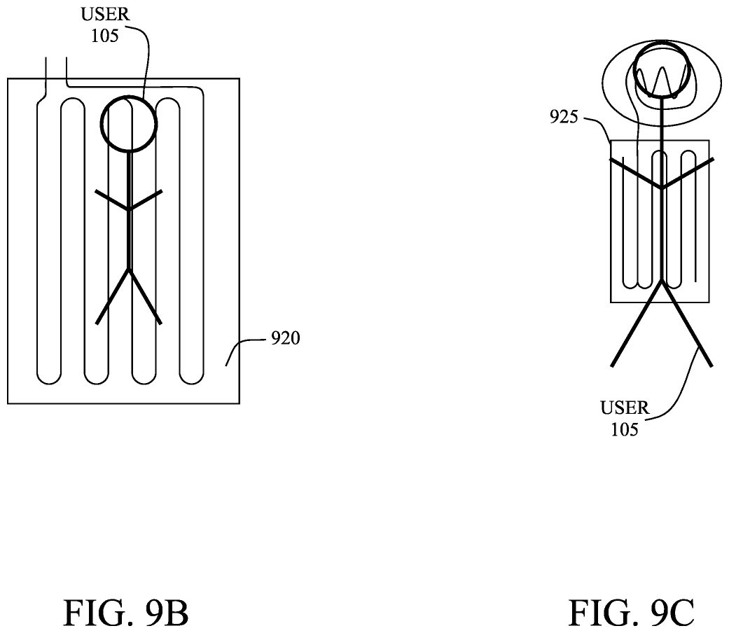

The illustrative pad contains layers that allow coolant to safely and uniformly flow through the pad without leaking or clogging, where outer layers of the pad provide comfort to the user and conceal the coolant layers. FIG. 9A illustrates an example cutaway view (e.g., top view) of the fluid containment layer of the mattress pad 910, where heat exchanger channels 915 allow for optimal removal of excess heat without the possibility of blockage. In one embodiment, the pad 910 may be built out of heat sealed thermoplastic polyurethane (TPU). The shape of the cooled location within the pad 910 is merely one example, and other options are available herein. For example, as shown in FIG. 9B, an example pad 920 may consist of a more rectangular arrangement for the entire space of a user 105 (e.g., head to toe and generally rectangular), while as shown in FIG. 9C, the cooling of a pad 925 may alternatively be more focused on the regions of the body women most often complain about (e.g., neck and back) rather than to cool the entire side of the bed evenly as in FIG. 9B.

As described above, the cooling hub may be attached to the mattress pad via hoses, and communicates with a wearable sensor (e.g., wristband) wirelessly. In this configuration, with reference again to FIG. 1, the controller 120 resides as part of the on-demand cooling system 130, which generally comprises the cooling hub and the mattress pad. However, the controller 120 may alternatively be co-located with the biometric sensors, such as on a smartwatch, or, more preferably, on a user's smartphone. That is, biometric sensors 110 (e.g., a wristband) may communicate wirelessly with a user's smartphone as the controller 120, which can log data in a remote database (e.g., cloud based in Cloud 140) or phone-based database for later access, and may make decisions on whether to actuate the on-demand cooling system 130 (e.g., independently, and/or with coordination with one or more cloud-based services).

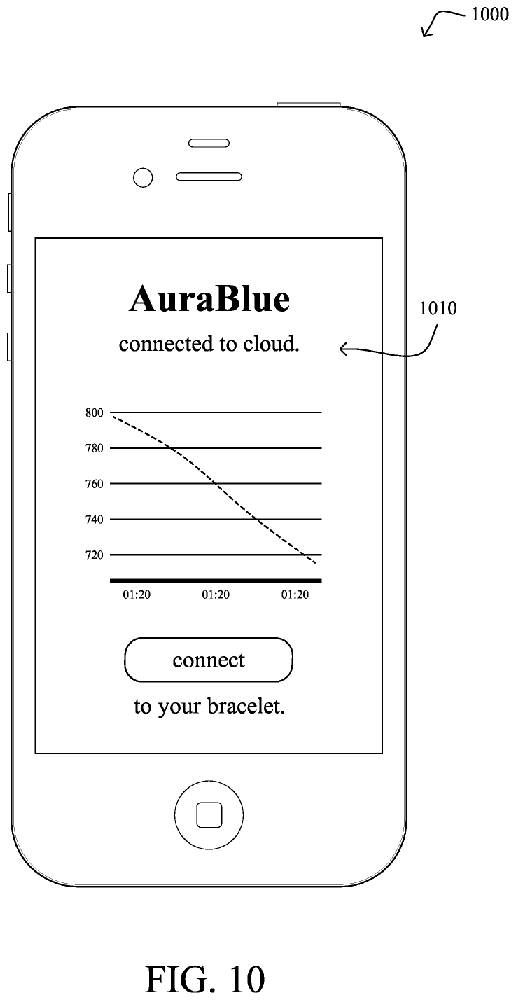

For instance, FIG. 10 illustrates an example user device (e.g., smartphone) 1000, with an associated mobile application/GUI 1010. The application (app) may be a portal to cloud-based services, and generally allows users to create accounts, relays data to the cloud, displays data as it comes in, provides sleep analytics, provides statistics around hot flash frequency and severity, and so on. As noted, in one embodiment, the app 1010 may independently determine when to control the on-demand cooling, or else may merely be a conduit to high-power cloud computation. Hybrid approaches are also conceived herein, such as where the app 1010 shares data with the cloud in order for various machine learning processes to create training data sets, classifiers, and other tools needed for the smartphone 1000 to be able to locally actuate the on-demand cooling system.

In particular, machine learning techniques may be utilized by one or more embodiments of the present disclosure to perform various portions of the techniques herein. In general, machine learning is concerned with the design and the development of techniques that take as input empirical data (such as biometric data, sleep patterns, and so on), and recognize complex patterns in these data. One very common pattern among machine learning techniques is the use of an underlying model M, whose parameters are optimized for minimizing the cost function associated to M, given the input data. For instance, in the context of classification, the model M may be a straight line that separates the data into two classes (e.g., labels) such that M=a*x+b*y+c and the cost function would be the number of misclassified points. The learning process then operates by adjusting the parameters a,b,c such that the number of misclassified points is minimal. After this optimization phase (or learning phase), the model M can be used very easily to classify new data points. Often, M is a statistical model, and the cost function is inversely proportional to the likelihood of M, given the input data.

Computational entities that rely on one or more machine learning techniques to perform a task for which they have not been explicitly programmed to perform are typically referred to as learning machines. In particular, learning machines are capable of adjusting their behavior to their environment. For example, a learning machine may dynamically make future predictions based on current or prior network measurements, may make control decisions based on the effects of prior control commands, etc.

For purposes of the techniques herein, a learning machine may construct a model (e.g., a supervised, un-supervised, or semi-supervised model) for use with the on-demand cooling actuation herein. Example machine learning techniques that may be used to construct and analyze such a model may include, but are not limited to, nearest neighbor (NN) techniques (e.g., k-NN models, replicator NN models, etc.), statistical techniques (e.g., Bayesian networks, etc.), clustering techniques (e.g., k-means, etc.), neural networks (e.g., reservoir networks, artificial neural networks, etc.), support vector machines (SVMs), or the like.

The on-demand cooling actuation herein, in particular, is illustratively based on a detection algorithm running on an application 1010 on the user's smartphone, and communicating (e.g., via Bluetooth low energy, WiFi, or wired protocols) with the on-demand cooling system to activate an appropriate response from the cooling system when necessary. However, any appropriate configuration may be embodied to perform the calculations and regressions necessary to predict temperature instability, sleep stage, skin conductivity, etc., and to enable prediction of oncoming temperature increases from the raw data received from the wearable sensors. That is, the performance may be on the cooling hub, within the Cloud, on the smartphone, or any combination of devices that are configured to assist in performing calculation and regressions, as well as data storage and transfer, accordingly.

Specifically, according to one or more embodiments of the techniques described herein, the system monitors the sensors and uses past and current data to predict the occurrences/onset of hot flashes and/or temperature instability in the user. When temperature instability is detected, the cooling system adjusts the flow of liquid (e.g., water) through the mattress pad to keep the user comfortable. In general, the reaction time for this process should be fast, predicting the need for cooling before the onset of a hot flash, at least in time to prevent the discomfort from awakening the user (e.g., within 60 seconds).

In particular, the combination of processing power within the sensor platform, hub, phone app, cloud resources, and so on, is used to implement a detection algorithm to predict hot flashes or other thermal stresses, and to react accordingly. This algorithm fuses data from several sensors, possibly including but not limited to skin conductance data (e.g., GSR sensors), photoplethysmogram (PPG) data, and motion data, for example, rapid eye motion (REM) data, as mentioned above. Each source of sensor data may be passed through one or more filters or transforms, possibly including but not limited to Kalman filters, low-pass filters, other frequency-based filters, Fourier transforms, and polynomial transforms. Following this, the resultant streams of data are fused using one or more neural nets (NNs) into values that correspond to biometric quantities, possibly including but not limited to peripheral vasodilation, heart rate, activity level, and sweating. These are then further processed using one or more filters, transforms, or neural nets into a single indicator of whether a hot flash or other temperature increase is occurring or about to occur.

For example, as described in greater detail below, various algorithms may be used to predict the onset of a hot flash or other thermal instability, such as exponential smoothing thresholds (e.g., slow moving curves versus faster moving averages) to focus on initial conductance spikes beyond a given threshold, ruling out other sources of noise. (Note that these spikes can readily exceed the baseline by 2-10×.) Other algorithms, such as the one mentioned above using one or more filters or transforms (e.g., Kalman filter->wavelet transform->neural net convolution->threshold for outputs correlating to hot flashes (outliers/spikes)) may also be used herein.

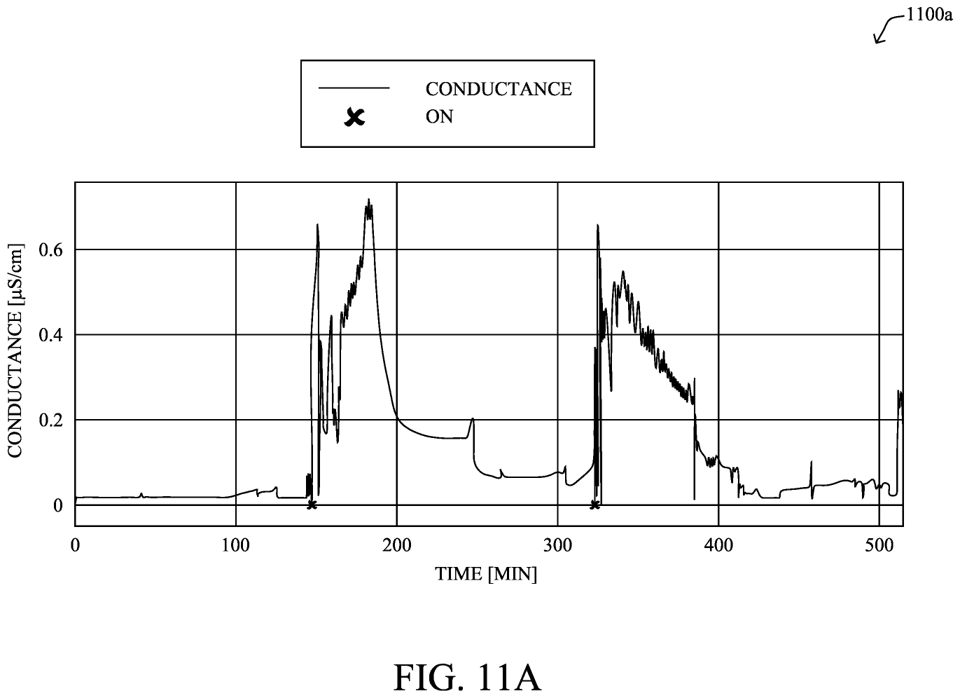

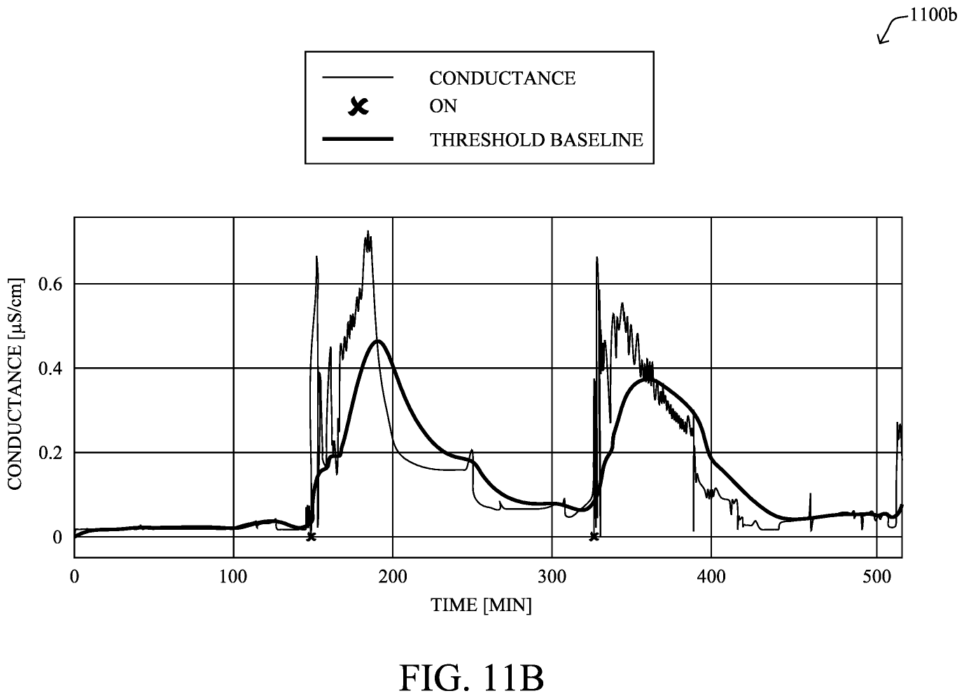

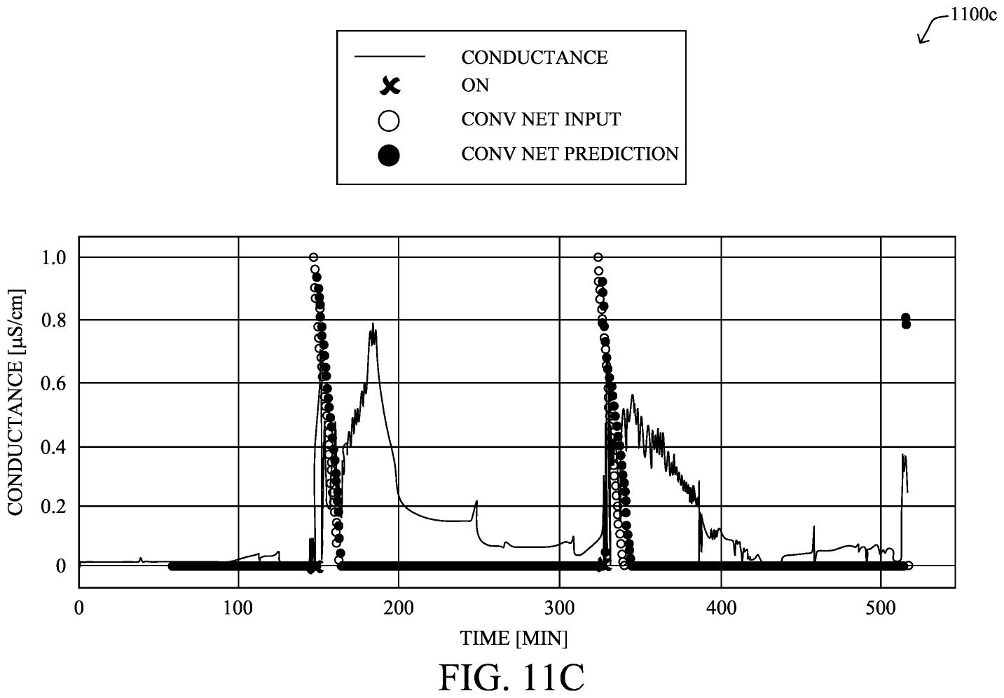

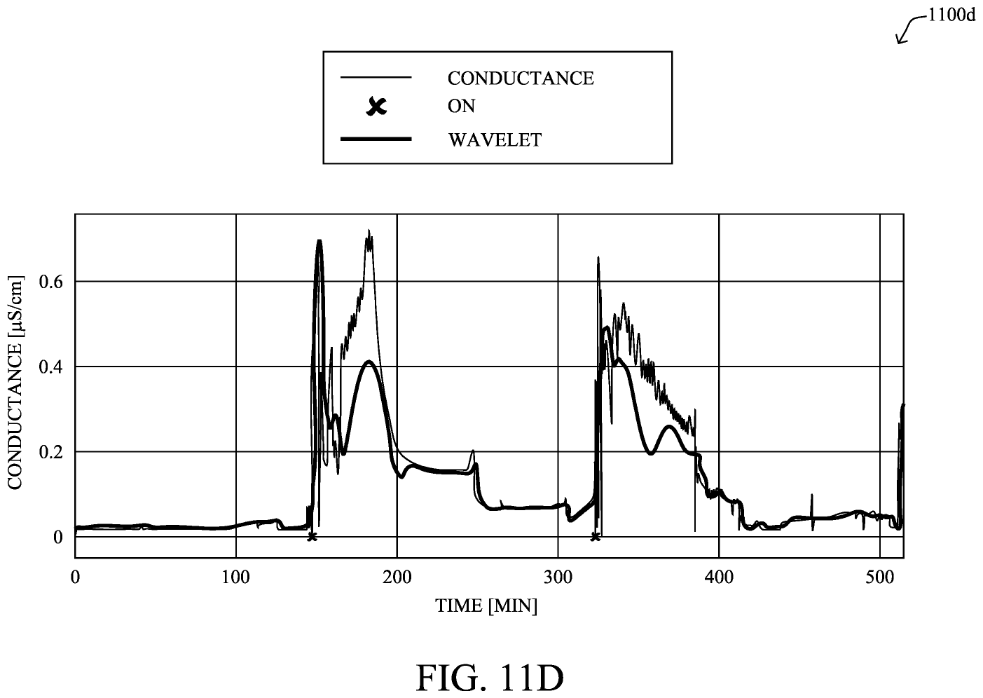

Regarding these “hot flash detecting” algorithm possibilities, detection of non-stationary and nonlinear events is generally based on trying to identify the presence of signal spikes in the presence of a relatively large amount of noise, while dealing with the fact that the spikes themselves may vary in profile. FIG. 11A illustrates a graph 1100a showing the difficulty of detecting hot flashes from conductance spikes, where unfiltered conductance is shown, punctuated by hot flash signaling button presses (“on”) by the user.

Though there are many approaches used by experts in event detection to detect corresponding activity (e.g., seismology, neurological behavior, etc.), many of these methods do not lend themselves easily to hot flash detection, since with hot flashes there is no negative conductance nor necessarily any periodicity (unlike seismic activity for instance). Techniques that are suitable, however, generally involve various filters/linear prediction (including Levinson), neural nets, templates, wavelet transform, simple thresholds, Markov models, higher order statistics, and distribution functions. Since templates can be approximated very well by/are essentially overfit neural nets, the discussion below generally does not address such techniques, essentially leaving thresholds, various filters, wavelets, and neural nets.

Using a threshold algorithm, for example, is generally very simple and easy to implement. For instance, using a basic exponential filter with a smoothing factor of, e.g., 0.001 to set a baseline, the algorithm detects when the signal exceeds this baseline by a set threshold. Illustratively, thresholds may generally range from 0.4 to 0.8, where increasing the threshold increases detection rate but also the amount of false positives. FIG. 11B illustrates a graph 1100b showing how this filter effectively selects for the initial spike: the threshold baseline is what the signal is compared to, such that the conductance measurements exceeding the baseline by a large enough amount to actuate the cooling only happens at the beginning of the hot flash spike, because the threshold baseline soon catches up.

An alternative algorithm, as noted above, which may be more complex but more effective, is now described in greater detail. In particular, the steps may generally be as follows:

1. Kalman filter:

Spring dashpot analogue with Kalman system noise dependent on how close to the baseline it is;

x here is the conductance minus the baseline, which is an estimate of the mode of the data created by repeatedly discarding outliers;

\newcommand{\vect}[1]{\hat{\mathbf{#1}}}

\begin{align*}

\vect{x} &=\begin{bmatrix} x \\ \dot{x} \end{bmatrix} \\

\dot{\vect{x}} &=\begin{bmatrix} \dot{x} \\−kx−b\dot{x} end{bmatrix}

\end{align*}

$$\hat{x} = [ \begin{matrix}x \\ \dot{x}\end{matrix} ] \\ \dot{\hat{x}} = [ \begin{matrix}\dot{x} \\ - kx - b \dot{x}\end{matrix} ] \\ k = 0.01 , b = 0.1 , R ( noise covariance ) = 0.4 ;$$

\begin{align*}

\mathbf{Q} &=\sigma_{a}\begin{bmatrix} \frac{\Delta t\hat{ }4} 4 & \frac{\Delta t\hat{ }3} 2 \\ \frac{\Delta t\hat{ }3} 2 & \Delta t\hat{ }2 \end{bmatrix} \\

\sigma_{a} &=\tau \abs{\frac 1 {x\hat{ }3}}

\end{align*}

$$Q = {σ}_{a} [ \begin{matrix}\frac{Δ {t}^{4}}{4} & \frac{Δ {t}^{3}}{2} \\ \frac{Δ {t}^{3}}{2} & Δ {t}^{2}\end{matrix} ] \\ {σ}_{a} = τ \frac{1}{{x}^{3}} \\ tau = 0.000001 ;$$

The purpose of tau is to make the system reactive to spikes near the baseline, but less tolerant of the secondary spikes which can be common in the aftermath of a hot flash.

2. Continuous wavelet transform:

Window size of 60 minutes;

Transform taken at 64 equally spaced time values in that window;

Transform taken with 16 sizes of wavelets, from 1 to the size of the window.

3. Convolutional neural net:

Batch normalization;

16 3×3 kernel convolutional;

24 3×9 kernel convolutional;

2×2 maxpool;

32 3×13 kernel convolutional;

16 3×5 kernel convolutional;

Fully connected layer with 8 outputs;

Fully connected layer with 1 output;

All ReLU activation except for the final output, which is sigmoid;

Uses an Adam optimizer with learning rate 0.001.

4. Simple threshold (e.g., 0.65) (after the neural net sigmoid) to say whether or not hot flash is currently happening.

Given the complexity of some of the hot flash profiles, the Kalman+wavelet+neural net should produce good results from training over a single hot flash. Over time, however, the computational system herein will be able to train/tweak parameters over a large dataset from the same user, allowing the algorithm to learn over time. (Learning for each user may require a hefty Lyapunov analysis to guarantee stability, so the techniques herein may perform system identification that could blend-together/optimize the results of different pre-trained neural nets.)

The Kalman+wavelet+neural net algorithm is notably robust against scaling—it looks for profiles, not thresholds. This is useful for looking at different users (e.g., women) with different levels of responses.

FIG. 11C illustrates an example output graph 1100c of the Kalman+wavelet+neural net algorithm above based on test conductance data, where the convolution network input and prediction correlate nicely with perceived hot flash conditions and associated measured conductance levels. Notably, this algorithm is remarkably stable, even across varying nets and parameters.

Still another algorithm that may be used herein is a Kalman+second generation (“2nd gen”) wavelets algorithm. In particular, a more discrete wavelet approach, called 2nd gen wavelets, is basically a very fast, very coarse wavelet transform that can be used in postprocessing of a dataset to identify key features for future algorithm refinement. A depiction of this algorithm's wavelet result on the same data set above is shown in graph 1100d of FIG. 11D.

Other algorithm for predicting the onset of a thermal instability (e.g., hot flash) based on monitored indicators of thermal stability (e.g., conductance sensors) in order to activate on-demand cooling systems (to counteract such predicted onsets) may be used herein, and those mentioned above are merely illustrative examples of possible algorithms. Further, while the discussion above references training based on sleep data, learning algorithms generally work better with more data, and thus may be collected throughout the day, even when the user is not benefiting from the cooling system (e.g., merely collecting data of hot flashes and corresponding biometric indicators). For instance, a user may wear the wearable sensors all day in order to better prepare the system for anticipating night-based hot flashes according to the user's own data/cycles. Still further, provisions may be made to the cooling system herein to provide daytime cooling mechanisms, such as cooling clothing, fans, air conditioning, and so on, and the techniques herein are not limited to sleep and/or nighttime.

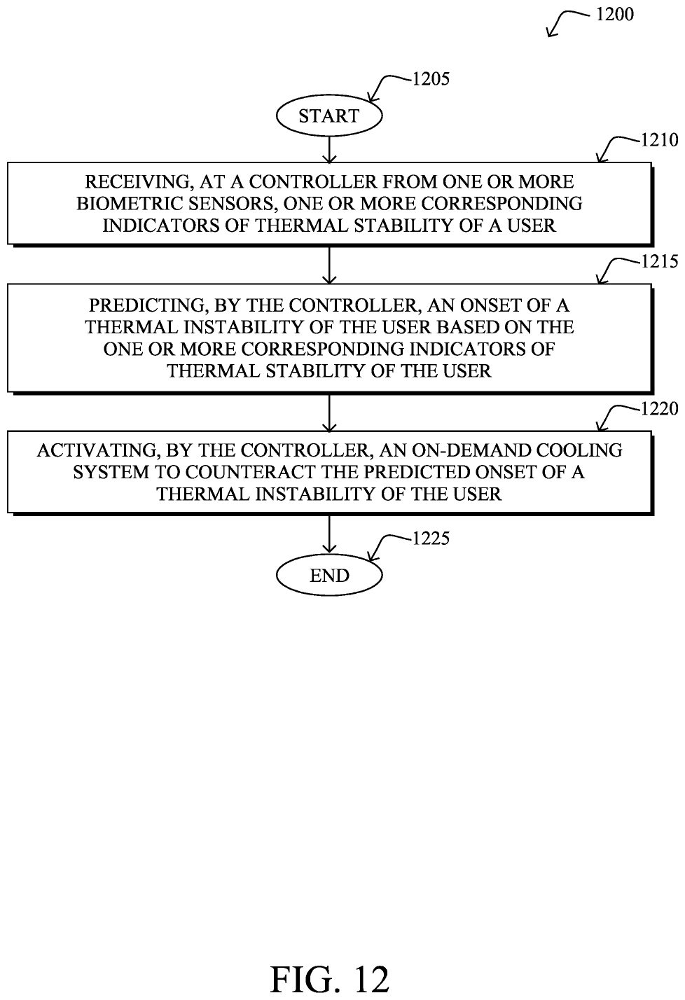

In closing, FIG. 12 illustrates an example simplified procedure 1200 for providing personal thermal stability control in accordance with one or more embodiments described herein. For example, a non-generic, specifically configured device (e.g., a single device or a system of devices, herein generally the “controller”) may perform procedure 1200 by having a processor execute stored instructions (e.g., a process) on the device(s) (e.g., in a memory). The procedure 1200 may start at step 1205, and continues to the following steps, as described generally in greater detail above.

In particular, in step 1210, the controller receives, from one or more biometric sensors, one or more corresponding indicators of thermal stability of a user. The controller may then, in step 1215, predict an onset of a thermal instability of the user based on the one or more corresponding indicators of thermal stability of the user. Accordingly, in step 1220, the controller may activate an on-demand cooling system to counteract the predicted onset of a thermal instability of the user, accordingly.

The simplified procedure 1200 then ends in step 1225. Note that other steps and details may also be included generally within the procedure above. For instance, the method may further comprise filtering the one or more corresponding indicators of thermal stability of the user to minimize data noise. The method may also further comprise deactivating the on-demand cooling system in response to counteracting the thermal instability of the user. Still further, the method may comprise collecting the one or more corresponding indicators of thermal stability of the user while the user is remotely located from the on-demand cooling system, where the one or more corresponding indicators of thermal stability of the user collected while the user is remotely located from the on-demand cooling system are to be used for training predictions of onsets of thermal instabilities of the user based on the one or more corresponding indicators of thermal stability of the user. Still other steps may be included above, and additional details, embodiments, implementations, and limitations may be applied to any of the steps mentioned above, as well.

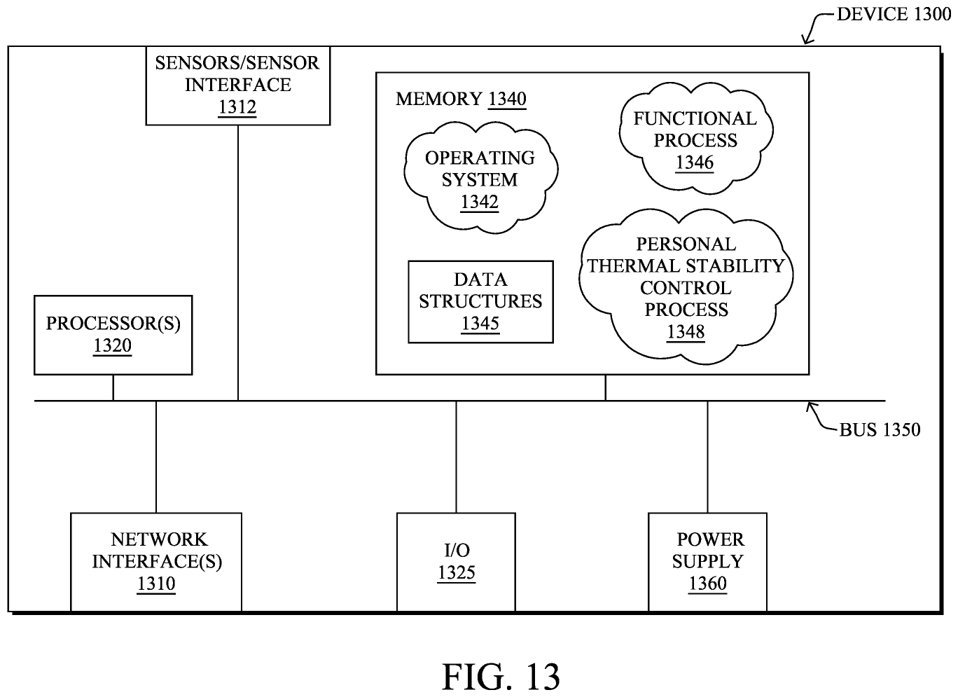

FIG. 13 is a schematic block diagram of an example simplified computing device (e.g., apparatus, controller, etc.) 1300 that may be used with one or more embodiments described herein, e.g., as any of the devices shown above, depending on functionality (e.g., wearable sensors, controller, hub, smartphone, etc.). The device may comprise one or more network interfaces 1310 (e.g., wired, wireless, etc.), at least one processor 1320, and a memory 1340 interconnected by a system bus 1350, as well as a power supply 1360 (e.g., battery, plug-in, etc.).

The network interface(s) 1310 contain the mechanical, electrical, and signaling circuitry for communicating data over links coupled to a network, e.g., providing a data connection between device 1300 and a local network or otherwise, such as the Internet. The network interfaces may be configured to transmit and/or receive data using a variety of different communication protocols. For example, interfaces 1310 may include wired transceivers, wireless transceivers, cellular transceivers, or the like, each to allow device 1300 to communicate information to and from a remote computing device or server over an appropriate network (e.g., the Cloud).

Various sensors 1312, including on-board sensors as well as interfaces to external sensors, may be included on the device 1300, such as accelerometers, skin conductance sensors, GSR sensors, PPG sensors, etc., as described above.

Device 1300 may also include an input/output (I/O) module 1325, such as for receiving user input (e.g., buttons, keyboards, graphical user interfaces or GUIs, etc.) or for providing user output (e.g., displays, lights, haptics, GUIs, etc.), as may be readily appreciated by those skilled in the art to embodiment one or more of the embodiments described in greater detail above.

The memory 1340 comprises a plurality of storage locations that are addressable by the processor 1320 and the network interfaces 1310 for storing software programs and data structures associated with the embodiments described herein. The processor 1320 may comprise hardware elements or hardware logic adapted to execute the software programs and manipulate the data structures 1345. An operating system 1342, portions of which are typically resident in memory 1340 and executed by the processor, functionally organizes the device by, among other things, invoking operations in support of software processes and/or services executing on the device. These software processes and/or services may comprise one or more functional processes 1346, and on certain devices, an illustrative “personal thermal stability control” process 1348, as described herein. Notably, functional processes 1346, when executed by processor(s) 1320, cause each particular device 1300 to perform the various functions corresponding to the particular device's purpose and general configuration. For example, a smartphone would be configured to operate as a smartphone, a server would be configured to operate as a server, a smartwatch would be configured to operate as smartwatch, and so on.

It will be apparent to those skilled in the art that other processor and memory types, including various computer-readable media, may be used to store and execute program instructions pertaining to the techniques described herein. Also, while the description illustrates various processes, it is expressly contemplated that various processes may be embodied as modules configured to operate in accordance with the techniques herein (e.g., according to the functionality of a similar process), and that more or fewer processes/modules may be included. Further, while the processes have been shown separately, those skilled in the art will appreciate that processes may be routines or modules within other processes. In addition, in certain embodiments, certain computations and/or operations may involve coordination between multiple computing devices, where the “process” comprises one or more such coordinated components.

Advantageously, the techniques herein provide personal thermal stability control. In particular, by cooling the user before and/or during a body temperature increase caused by factors including but not limited to hot flashes, fevers or other illnesses, environmental temperature fluctuations, vasomotor symptoms and the like, the present disclosure increases sleep quality, which can be measured by the frequency or duration of waking incidents for the user or a partner sharing the bed, the number of incidents where the user leaves the bed during the night, number of times the covers need to be adjusted, and any other biometric measure of sleep quality and restfulness.

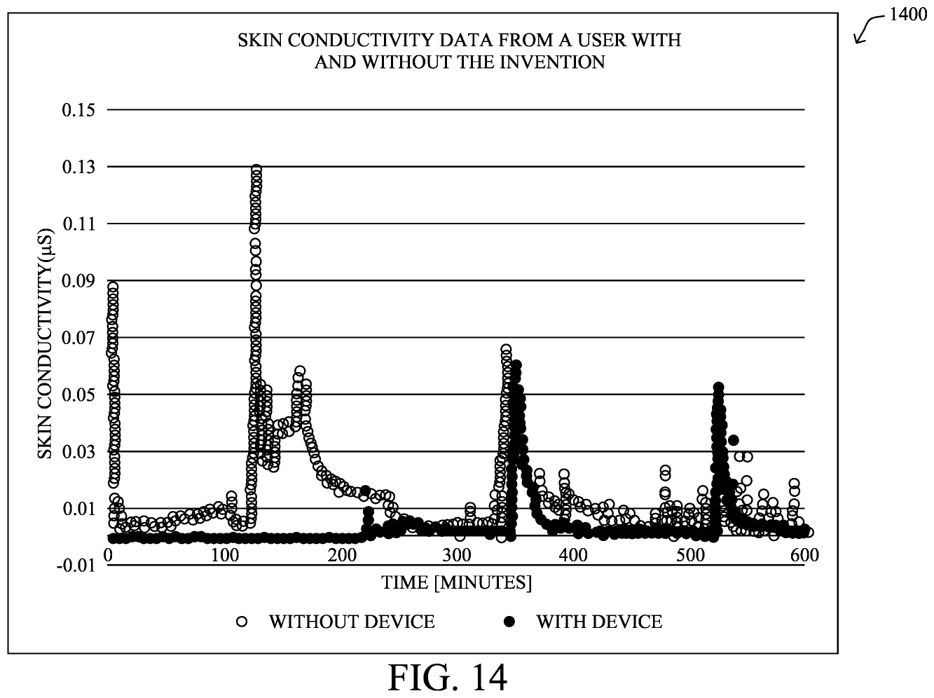

An example of the techniques herein increasing sleep quality by decreasing sweating amplitude and duration is shown in FIG. 14, where the graph 1400 shows (as a scatterplot) data that was collected from a woman experiencing hot flashes on a night when they were using a system according to one or more embodiments described herein, and on a night where they were not. The system herein successfully decreased the magnitude and severity of sweating compared to the control night, as measured through skin conductivity, and similar results have been obtained on other patients. In particular, as shown in FIG. 14, the Y-axis represents skin conductivity, a metric relating to hot flash induced sweating, which can be used to show the severity and duration of a hot flash. The X-axis represents the time in minutes. For both datasets, data was collected from the same individual over the duration of an entire night. The “Without Device” dataset displays higher spikes that last longer than the “With Device” dataset. Traces around 125-175 min on the X-axis show that the device helped the user avoid a hot-flash induced sweating episode in early sleep, and traces from 350-400 min on the X-axis show that when there was an episode, its duration was less with the device than without.

Notably, the embodiments herein provide various advantages over competitive solutions that attempt to cool users. For example, while studies have shown that women's core temperatures may fluctuate slightly during hot flashes, the variations are borderline undetectable. As such, rather than being concerned specifically with current and/or future body temperature, whether predicting or trying to change body temperatures, the techniques herein are more directed to measuring and counteracting the symptoms of hot flash symptoms, for example, such as measuring conductance (e.g., sweatiness) and correspondingly preventing vasodilation (one of the key underlying causes of hot flash symptoms). (Notably, the techniques herein may use body temperature as one of many possible inputs to the algorithms above, but are not merely dependent on it, and in general, do not consider body temperature a primary indicator). In addition, the techniques herein are collecting and using data from many past hot flashes to train the system, and to inform the system what to do with a predicted and/or current hot flash. In other words, more than merely reacting to a high body temperature, the techniques herein use other (better) indicators and a trained model to predict the onset of a hot flash, before the user is already uncomfortable. Still further, the techniques herein intelligently minimize noise (particularly in user conductance) through careful sensor placement and hardware and software selection, since these can readily exceed the raw signal, but can be subtly differentiated from a hot flash, accordingly.

While the present disclosure has illustrated various embodiments and specific implementations, other configurations may be made within the scope of the invention. For instance, while certain materials may have been shown or implied for each component, other suitable materials may be used. Furthermore, while certain shapes or designs of the components have been shown and described, functionally similar designs may also be utilized herein. Moreover, while components of the present disclosure may be described separately and in separate figures, certain components from each embodiment may be incorporated into each other embodiment, and the components shown in each of the illustrations are not meant to be mutually exclusive. That is, various combinations of components may be made with the scope of the present disclosure by combining the described components in useful manners.

Additionally, while the description above often refers to women and hot flashes, the techniques herein may be used by any gender and for any thermal instability.

It should also be noted that any steps shown and/or described in any procedure(s) or discussions above are merely examples for illustration, and certain other steps may be included or excluded as desired. Further, while a particular order of the steps may have been discussed and/or shown, this ordering is merely illustrative, and any suitable arrangement of the steps may be utilized without departing from the scope of the embodiments herein.

Furthermore, in the detailed description, numerous specific details are set forth in order to provide a thorough understanding of some embodiments. However, it will be understood by persons of ordinary skill in the art that some embodiments may be practiced without these specific details. In other instances, well-known methods, procedures, components, units and/or circuits have not been described in detail so as not to obscure the discussion.

As a general account of the description above, in one embodiment, an illustrative system according to one or more embodiments of the present disclosure may comprise: an on-demand cooling system; one or more biometric sensors configured to monitor one or more corresponding indicators of thermal stability of a user; and a controller configured to: a) receive the one or more corresponding indicators of thermal stability of the user; b) predict an onset of a thermal instability of the user based on the one or more corresponding indicators of thermal stability of the user; and c) activate the on-demand cooling system to counteract the predicted onset of a thermal instability of the user.

In one embodiment, the thermal instability corresponds to a hot flash.

In one embodiment, the one or more biometric sensors are embodied within a wearable device selected from a group consisting of: a watch; a bracelet; an anklet; a wristband; a glove; a partial glove; fingertip straps; and an eye mask.

In one embodiment, the one or more biometric sensors are selected from a group consisting of: a galvanic skin resistance (GSR) sensor; a photoplethysmogram (PPG) sensor; a motion detector; an accelerometer; a gyroscope; a rapid eye movement (REM) sensor; a conductance sensor; a sweat sensor; a heart rate sensor; and a blood flow sensor.

In one embodiment, the controller is further configured to filter the one or more corresponding indicators of thermal stability of the user to minimize data noise.

In one embodiment, one or more of the one or more biometric sensors comprise a noise minimization mechanism. In one embodiment, the noise minimization mechanism comprises a physical mechanism configured to maintain consistent pressure of a corresponding biometric sensor against the user. In one embodiment, the noise minimization mechanism comprises an electronic circuit to assist in a determination of pressure of a corresponding biometric sensor against the user.

In one embodiment, the controller is configured to predict an onset of a thermal instability of the user based on early detection or anticipation of one or more of peripheral vasodilation, heart rate, activity level, and sweating of the user based on the one or more corresponding indicators of thermal stability of the user.

In one embodiment, the controller is configured to predict an onset of a thermal instability of the user based on detection of an increase spike in one or more of the one or more corresponding indicators of thermal stability of the user.

In one embodiment, the controller is further configured to deactivate the on-demand cooling system in response to counteracting the thermal instability of the user.

In one embodiment, the on-demand cooling system comprises a liquid-based bed-based cooling system. In one embodiment, the liquid-based bed-based cooling system is selected from a group consisting of: a mattress; mattress pad; and a blanket. In one embodiment, the liquid-based bed-based cooling system comprises a reservoir of liquid pre-cooled prior to activation of the on-demand cooling system. In one embodiment, the liquid-based bed-based cooling system is shaped to focus cooled liquid to an area corresponding to a neck and a back of the user.

In one embodiment, the on-demand cooling system comprises an air-based cooling system.

In one embodiment, the controller is further configured to collect the one or more corresponding indicators of thermal stability of the user while the user is remotely located from the on-demand cooling system, the one or more corresponding indicators of thermal stability of the user collected while the user is remotely located from the on-demand cooling system used for training predictions of onsets of thermal instabilities of the user based on the one or more corresponding indicators of thermal stability of the user.

In one embodiment, at least a portion of the controller comprises a smartphone application.

Moreover, in one embodiment, an illustrative method according to one or more embodiments of the present disclosure may comprise: receiving, at a controller from one or more biometric sensors, one or more corresponding indicators of thermal stability of a user; predicting, by the controller, an onset of a thermal instability of the user based on the one or more corresponding indicators of thermal stability of the user; and activating, by the controller, an on-demand cooling system to counteract the predicted onset of a thermal instability of the user.

Also, in one embodiment, an illustrative tangible, non-transitory, computer-readable medium according to one or more embodiments of the present disclosure may store program instructions that cause a computer (processing device) to execute a process comprising: receiving, from one or more biometric sensors, one or more corresponding indicators of thermal stability of a user; predicting an onset of a thermal instability of the user based on the one or more corresponding indicators of thermal stability of the user; and causing a signal to activate an on-demand cooling system to counteract the predicted onset of a thermal instability of the user.

Further, in one embodiment, an illustrative apparatus according to one or more embodiments of the present disclosure may comprise: a processor configured to execute one or more processes; a communication interface configured to communicate via one or more wired and/or wireless communication protocols; and a memory configured to store a process executable by the processor, the process, when executed, configured to perform a process comprising: receiving, from one or more biometric sensors, one or more corresponding indicators of thermal stability of a user; predicting an onset of a thermal instability of the user based on the one or more corresponding indicators of thermal stability of the user; and activating, by the controller, an on-demand cooling system to counteract the predicted onset of a thermal instability of the user.

The foregoing description has been directed to specific embodiments. It will be apparent, however, that other variations and modifications may be made to the described embodiments, with the attainment of some or all of their advantages. For instance, it is expressly contemplated that certain components and/or elements described herein can be implemented as software being stored on a tangible (non-transitory) computer-readable medium (e.g., disks/CDs/RAM/EEPROM/etc.) having program instructions executing on a computer, hardware, firmware, or a combination thereof. Accordingly this description is to be taken only by way of example and not to otherwise limit the scope of the embodiments herein. Therefore, it is the object of the appended claims to cover all such variations and modifications as come within the true intent and scope of the embodiments herein.

What is claimed is:

1. A system, comprising:
an on-demand cooling system;
one or more biometric sensors configured to monitor one or more corresponding indicators of thermal stability of a user; and
a controller configured to:
a) receive the one or more corresponding indicators of thermal stability of the user;
b) predict an onset of a thermal instability of the user based on the one or more corresponding indicators of thermal stability of the user; and
c) activate the on-demand cooling system to counteract the predicted onset of a thermal instability of the user.

2. The system as in claim 1, wherein the thermal instability corresponds to a hot flash.

3. The system as in claim 1, wherein the one or more biometric sensors are embodied within a wearable device selected from a group consisting of: a watch; a bracelet; an anklet; a wristband; a glove; a partial glove; fingertip straps; and an eye mask.

4. The system as in claim 1, wherein the one or more biometric sensors are selected from a group consisting of: a galvanic skin resistance (GSR) sensor; a photoplethysmogram (PPG) sensor; a motion detector; an accelerometer; a gyroscope; a rapid eye movement (REM) sensor; a conductance sensor; a sweat sensor; a heart rate sensor; and a blood flow sensor.

5. The system as in claim 1, wherein the controller is further configured to filter the one or more corresponding indicators of thermal stability of the user to minimize data noise.

6. The system as in claim 1, wherein one or more of the one or more biometric sensors comprise a noise minimization mechanism.

7. The system as in claim 6, wherein the noise minimization mechanism comprises a physical mechanism configured to maintain consistent pressure of a corresponding biometric sensor against the user.

8. The system as in claim 6, wherein the noise minimization mechanism comprises an electronic circuit to assist in a determination of pressure of a corresponding biometric sensor against the user.

9. The system as in claim 1, wherein the controller is configured to predict an onset of a thermal instability of the user based on early detection or anticipation of one or more of peripheral vasodilation, heart rate, activity level, and sweating of the user based on the one or more corresponding indicators of thermal stability of the user.

10. The system as in claim 1, wherein the controller is configured to predict an onset of a thermal instability of the user based on detection of an increase spike in one or more of the one or more corresponding indicators of thermal stability of the user.

11. The system as in claim 1, wherein the controller is further configured to deactivate the on-demand cooling system in response to counteracting the thermal instability of the user.

12. The system as in claim 1, wherein the on-demand cooling system comprises a liquid-based bed-based cooling system.

13. The system as in claim 12, wherein the liquid-based bed-based cooling system is selected from a group consisting of: a mattress; mattress pad; and a blanket.

14. The system as in claim 12, wherein the liquid-based bed-based cooling system comprises a reservoir of liquid pre-cooled prior to activation of the on-demand cooling system.

15. The system as in claim 12, wherein the liquid-based bed-based cooling system is shaped to focus cooled liquid to an area corresponding to a neck and a back of the user.

16. The system as in claim 1, wherein the on-demand cooling system comprises an air-based cooling system.

17. The system as in claim 1, wherein the controller is further configured to collect the one or more corresponding indicators of thermal stability of the user while the user is remotely located from the on-demand cooling system, the one or more corresponding indicators of thermal stability of the user collected while the user is remotely located from the on-demand cooling system used for training predictions of onsets of thermal instabilities of the user based on the one or more corresponding indicators of thermal stability of the user.

18. The system as in claim 1, wherein at least a portion of the controller comprises a smartphone application.

19. A method, comprising:
receiving, at a controller from one or more biometric sensors, one or more corresponding indicators of thermal stability of a user;
predicting, by the controller, an onset of a thermal instability of the user based on the one or more corresponding indicators of thermal stability of the user; and
activating, by the controller, an on-demand cooling system to counteract the predicted onset of a thermal instability of the user.

20. A tangible, non-transitory, computer-readable medium storing program instructions that cause a computer on a processing device to execute a process, the process comprising:
receiving, from one or more biometric sensors, one or more corresponding indicators of thermal stability of a user;
predicting an onset of a thermal instability of the user based on the one or more corresponding indicators of thermal stability of the user; and
causing a signal to activate an on-demand cooling system to counteract the predicted onset of a thermal instability of the user.
